(19) United States
(12) Patent Application Publication
(10) Pub. No.: US 2009/0081743 A1
(43) Pub. Date: Mar. 26, 2009

(54) TRANSPORTABLE ALGAE BIODIESEL SYSTEM

(76) Inventors: David A. Hazelbeck, El Cajon, CA (US); Eric H. Dunlop, Paradise (AU); Holger H. Streckert, San Diego, CA (US)

Correspondence Address: NYDEGGER & ASSOCIATES, 348 OLIVE STREET, SAN DIEGO, CA, 92103, US

(21) Appl. No.: 11/860,341

(22) Filed: Sep. 24, 2007

(51) Int. Cl. C12P 7/04; C12M 3/00; C12N 5/02; C12P 7/00

(52) U.S. Cl. 435/157; 435/132; 435/293.1

(57) ABSTRACT

A portable system and method for producing biofuel from algae are disclosed. In the portable system, a chemostat and a plug flow reactor formed from plastic bladders are interconnected. Further, an algae separator is in fluid communication with the plug flow reactor for removing algae cells. Also, the system includes a device for processing biofuel from the algae cells. Importantly, the system includes a temperature controller to maintain desired temperatures in the chemostat and plug flow reactor for algae growth and intracellular algae production. In order to further support algae cell growth, the system includes a device for capturing carbon dioxide and delivering the carbon dioxide to the chemostat.

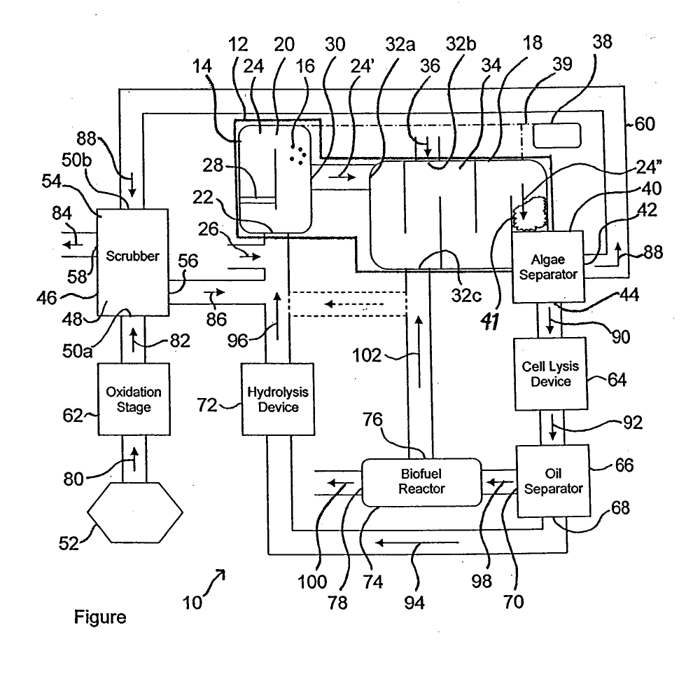

### FIELD OF THE INVENTION

The present invention pertains generally to processes for producing biofuel from oil in algae. More particularly, the present invention pertains to a portable system that grows algae cells having a high oil content and synthesizes the oil into biofuel. The present invention is particularly, but not exclusively, useful as a portable system and method that utilizes available carbon in waste and pollution to grow algae for processing into biofuel.

### BACKGROUND OF THE INVENTION

As worldwide petroleum deposits decrease, there is rising concern over shortages and the costs that are associated with the production of hydrocarbon products. As a result, alternatives to products that are currently processed from petroleum are being investigated. In this effort, biofuels such as biodiesel have been identified as a possible alternative to petroleum-based transportation fuels. In general, biodiesel is a fuel comprised of mono-alkyl esters of long chain fatty acids derived from plant oils or animal fats. In industrial practice, biodiesel is created when plant oils or animal fats are reacted with an alcohol, such as methanol.

For plant-derived biofuel, solar energy is first transformed into chemical energy through photosynthesis. The chemical energy is then refined into a usable fuel. Currently, the process involved in creating biofuel from plant oils is expensive relative to the process of extracting and refining petroleum. It is possible, however, that the cost of processing a plant-derived biofuel could be reduced by maximizing the rate of growth of the plant source. Because algae is known to be one of the most efficient plants for converting solar energy into cell growth, it is of particular interest as a biofuel source. However, current algae processing methods have failed to result in a cost effective algae-derived biofuel.

In overview, the biochemical process of photosynthesis provides algae with the ability to convert solar energy into chemical energy. During cell growth, this chemical energy is used to drive synthetic reactions, such as the formation of sugars or the fixation of nitrogen into amino acids for protein synthesis. Excess chemical energy is stored in the form of fats and oils as triglycerides. Thus, the creation of oil in algae only requires sunlight, carbon dioxide and the nutrients necessary for formation of triglycerides. Nevertheless, with the volume requirements for a fuel source, the costs associated with the inputs are high.

In certain applications, costs associated with conventional fuels are also quite high. Specifically, forward military bases and remote exploratory camps experience high fuel costs due to the expenses involved in delivering fuel. Also, ships typically must travel to ports simply to refuel. Therefore, fuel costs can be reduced if fuel is produced at the desired site, rather than transported to the desired site.

In light of the above, it is an object of the present invention to provide a system and method for producing biofuel from algae which reduces input costs. For this purpose, a number of systems have been developed, such as those disclosed in co-pending U.S. patent application Ser. No. ______ for an invention entitled “High Efficiency Separations to Recover Oil from Microalgae,” which is filed concurrently herewith, co-pending U.S. patent application Ser. No. 11/549,532 for an invention entitled “Photosynthetic Oil Production in a Two-Stage Reactor” filed Oct. 13, 2006, co-pending U.S. patent application Ser. No. 11/549,541 for an invention entitled “Photosynthetic Carbon Dioxide Sequestration and Pollution Abatement” filed Oct. 13, 2006, co-pending U.S. patent application Ser. No. 11/549,552 for an invention entitled “High Photoefficiency Microalgae Bioreactors” filed Oct. 13, 2006, and co-pending U.S. patent application Ser. No. 11/549,561 for an invention entitled “Photosynthetic Oil Production with High Carbon Dioxide Utilization” filed Oct. 13, 2006. All aforementioned co-pending U.S. patent applications are assigned to the same assignee as the present invention, and are hereby incorporated by reference. Another object of the present invention is to provide a portable recycling system for feeding oil harvesting byproducts back to the conduit where high oil content algae is grown. Still another object of the present invention is to provide a portable system for supplying nutrients to algae cells in the form of processed algae cell matter. Another object of the present invention is to provide a portable system for recycling the glycerin byproduct from the creation of biofuel as a source of carbon to foster further oil production in algae cells. Another object of the present invention is to provide a portable system for processing oil from algae that defines a flow path for continuous movement of the algae and its processed derivatives. Yet another object of the present invention is to provide a portable system and method for producing biofuel from algae with high oil content that is simple to implement, easy to use, and comparatively cost effective.

### SUMMARY OF THE INVENTION

In accordance with the present invention, a portable system is provided for efficiently producing biofuel from algae. For this purpose, the system utilizes a collapsible plastic bladder that forms a chemostat and a plug flow reactor. Structurally, the chemostat defines a conduit for growing algae cells. The chemostat's conduit includes input ports for feeding material into the conduit as well as an output port. Further, the plug flow reactor defines a conduit for fostering oil production within the algae cells. For the present invention, the plug flow reactor has an input port that is positioned to receive material from the output port of the chemostat. Also the system is provided with a temperature control that monitors and maintains the temperature within the conduits.

In addition to the plastic bladder and temperature control, the system includes an algae separator. Specifically, the algae separator is positioned in fluid communication with the plug flow reactor to remove an algae cell concentrate from the plug flow reactor's conduit. Structurally, the algae separator includes an outlet for the remaining effluence which is in fluid communication with the input port of the chemostat. Further, the system includes a device for lysing algae cells to unbind oil from the algae cells. For purposes of the present invention, the lysing device is positioned to receive algae cells from the algae separator.

Downstream of the lysing device, the system includes an oil separator that receives the lysed cells and withdraws the oil from remaining cell matter. For purposes of the present invention, the oil separator has an outlet for the remaining cell matter which is in fluid communication with the input port of the chemostat. Further, the system may include a hydrolyzing device interconnected between the oil separator and the chemostat. In addition to the cell matter outlet, the oil separator includes an outlet for the oil. For the present invention, the system includes a biofuel reactor that is in fluid communication with the outlet for oil. In a known process, the biofuel reactor reacts an alcohol with the oil to synthesize biofuel and, as a byproduct, glycerin. Structurally, the biofuel reactor includes an exit for the glycerin that is in fluid communication with the input port of the plug flow reactor.

For purposes of the present invention, the system includes a scrubber having a chamber for receiving a pollutant-contaminated fluid stream and a scrubber solution. Typically, the fluid stream comprises flue gas from a combustion source, such as a power plant or incinerator. Further, the scrubber solution is typically a caustic or sodium bicarbonate. Downstream of the algae separator, the system includes a channel for recycling an effluence from the plug flow reactor to the scrubber for reuse as the scrubber solution.

In operation, the flue gas from the power plant is flowed through the chamber of the scrubber. At the same time, the scrubber solution is sprayed into the scrubber chamber to capture the pollutants in the flue gas. The scrubber solution with the entrapped pollutants is then delivered to the chemostat through its input port. Also, a nutrient mix may be fed into the chemostat through the input port to form, along with the scrubber solution, a medium for growing algae cells. As the medium circulates through the conduit of the chemostat, the algae cells grow using solar energy and converting the pollutants and other nutrients to cell matter. Preferably, a continuous flow of the medium washes the algae cells and constantly removes them from the chemostat as overflow. In the plug flow reactor, the algae cells are treated to produce intracellular oil. Thereafter, the algae separator removes the algae cells from the remaining effluence in the plug flow reactor.

Then, the effluence is recycled through a channel back to the scrubber for reuse as the scrubber solution. At the same time, the algae cells are delivered to the cell lysis apparatus. At the cell lysis device, the cells are lysed, preferably with steam, to unbind the oil from the remaining cell matter. This unbound cell material is received by the oil separator from the cell lysis device. Next, the oil separator withdraws the oil from the remaining cell matter and effectively forms two streams of material. The stream of remaining cell matter is transferred to the hydrolysis device where the cell matter is broken into small units which are more easily absorbed by algae cells during cell growth. Thereafter, the hydrolyzed cell matter is delivered to the chemostat to serve as a source of nutrition for the algae cells growing therein. At the same time, the stream of oil is transmitted from the oil separator to the biofuel reactor. In the biofuel reactor, the oil is reacted with an alcohol to form biofuel and a glycerin byproduct. The glycerin byproduct is fed back into the plug flow reactor to serve as a source of carbon for the algae cells therein during the production of intracellular oil.

### BRIEF DESCRIPTION OF THE DRAWINGS

The novel features of this invention, as well as the invention itself, both as to its structure and its operation, will be best understood from the accompanying drawing, taken in conjunction with the accompanying description, in which the FIGURE is a schematic view of the portable system for producing biofuel from algae in accordance with the present invention.

### DESCRIPTION OF THE PREFERRED EMBODIMENTS

Referring to the FIGURE, a portable system for producing biofuel from algae in accordance with the present invention is shown and generally designated 10. As shown, the system 10 includes a plastic bladder 12 that forms at least one chemostat 14 for growing algae cells (exemplary cells depicted at 16) and a plug flow reactor 18 for treating the algae cells 16 to trigger cell production of triglycerides. For purposes of the present invention, the plastic bladder 12 is easily collapsed and stored to facilitate transportation to, and assembly of the system 10, at remote locations.

As shown in the FIGURE, the chemostat 14 includes a conduit 20. As further shown, the conduit 20 is provided with an input port 22 for receiving a medium 24. For purposes of the present invention, the input port 22 is also in communication with a reservoir (not illustrated) holding a nutrient mix (indicated by arrow 26). Preferably, the nutrient mix 26 includes phosphorous, nitrogen, sulfur and numerous trace elements necessary to support algae growth. Further, the chemostat 14 is provided with an Archimedes screw 28 for causing the medium 24 and the nutrient mix 26 to continuously circulate around the conduit 20 at a predetermined fluid flow velocity. Also, each conduit 20 is provided with an output port 30 in communication with the plug flow reactor 18.

As shown, the plug flow reactor 18 includes an input port 32a for receiving overflow medium (indicated by arrow 24′) with algae cells 16 from the output port 30 of the chemostat 14. As further shown, the plug flow reactor 18 includes a conduit 34 for passing the medium 24″ with algae cells 16 downstream. The flow rate of the medium 24″ is due solely to gravity and the force of the incoming overflow medium 24′ from the chemostat 14. Preferably, the plug flow reactor 18 has a substantially fixed residence time of about one to four days. For purposes of the present invention, the system 10 is provided with a reservoir (not shown) that holds a modified nutrient mix (indicated by arrow 36). Further, the conduit 34 is provided with an input port 32b for receiving the modified nutrient mix 36. In order to manipulate the cellular behavior of algae cells 16 within the plug flow reactor 18, the modified nutrient mix 36 may contain a limited amount of a selected constituent, such as nitrogen or phosphorous. For instance, the nutrient mix 36 may contain no nitrogen. Alternatively, the algae cells 16 may exhaust nutrients such as nitrogen or phosphorous in the nutrient mix 26 at a predetermined point in the plug flow reactor 18. By allowing such nutrients to be exhausted, desired behavior in the algae cells 16 can be caused without adding a specific modified nutrient mix 36. Further, simply water can be added through the modified nutrient mix 36 to compensate for evaporation. In addition to input ports 32a and 32b, the conduit 34 is further provided with an input port 32c to receive other matter.

For purposes of the present invention, the system 10 further includes a temperature control 38 that is connected to the chemostat 14 and the plug flow reactor 18 via leads 39. Specifically, the temperature control 38 monitors the temperature of the medium 24 and heats or cools the medium 24 as needed to provide a suitable environment for algae growth.

As shown in the FIGURE, the system 10 also includes an algae separator 40 for removing the algae cells 16 from the plug flow reactor 18. Specifically, the algae cells 16 form an algae cell concentrate 41 that is separated by the algae separator 40 from the medium 24″ and the remaining nutrients therein through flocculation and/or filtration. As further shown, the algae separator 40 includes an effluence outlet 42 and an algae cell outlet 44.

For further purposes of the present invention, the system 10 includes a scrubber 46 for scrubbing a pollutant-contaminated fluid stream. Specifically, the scrubber 46 includes a chamber 48 and an input port 50a for receiving flue gas from a combustion source such as a power plant or incinerator 52 and a scrubber solution 54. Typically, the flue gas includes pollutants such as carbon dioxide, sulfur oxides, and/or nitrogen oxides. Also, the scrubber solution 54 typically comprises sodium phosphate or sodium bicarbonate. As further shown, the scrubber 46 includes a solution outlet 56 and a gas outlet 58. As illustrated, the solution outlet 56 is in fluid communication with the input port 22 of the chemostat 14. For purposes of the present invention, the scrubber 46 includes a solution input port 50b in the scrubber chamber 48. Further, the system 10 includes a channel 60 providing fluid communication between the effluence outlet 42 and the scrubber 46 through the solution input port 50b. Also, the system 10 includes an oxidation stage 62 for oxidizing pollutants in the flue gas to facilitate their removal from the flue gas. As shown, the oxidation stage 62 is interconnected between the carbon source 52 and the scrubber 46.

In the FIGURE, the system 10 includes a cell lysis apparatus 64 that receives algae cells 16 from the algae outlet 44 of the algae separator 40. As shown, the cell lysis apparatus 64 is in fluid communication with an oil separator 66. For purposes of the present invention, the oil separator 66 is provided with two outlets 68, 70. As shown, the outlet 68 is connected to a hydrolysis apparatus 72. Further, the hydrolysis apparatus 72 is connected to the input port 22 in the conduit 20 of the chemostat 14.

Referring back to the oil separator 66, it can be seen that the outlet 70 is connected to a biofuel reactor 74. It is further shown that the biofuel reactor 74 includes two exits 76, 78. For purposes of the present invention, the exit 76 is connected to the input port 32c in the conduit 34 of the plug flow reactor 18. Additionally or alternatively, the exit 76 may be connected to the input port 22 in the chemostat 14. Further, the exit 78 may be connected to a tank or reservoir (not shown) for purposes of the present invention.

In operation of the present invention, pollutant-contaminated flue gas (indicated by arrow 80) is directed from the carbon source 52 to the oxidation stage 62. At the oxidation stage 62, nitrogen monoxide in the flue gas 80 is oxidized by nitric acid or by other catalytic or non-catalytic technologies to improve the efficiency of its subsequent removal. Specifically, nitrogen monoxide is oxidized to nitrogen dioxide. Thereafter, the oxidized flue gas (indicated by arrow 82) is delivered from the oxidation stage 62 to the scrubber 46. Specifically, the oxidized flue gas 82 enters the chamber 48 of the scrubber 46 through the input port 50a. Upon the entrance of the oxidized flue gas 82 into the chamber 48, the scrubber solution 54 is sprayed within the chamber 48 to absorb, adsorb or otherwise trap the pollutants in the oxidized flue gas 82 as is known in the field of scrubbing. With its pollutants removed, the clean flue gas (indicated by arrow 84) exits the scrubber 46 through the gas outlet 58. At the same time, the scrubber solution 54 and the pollutants exit the scrubber 46 through the solution outlet 56.

After exiting the scrubber 46, the scrubber solution 54 and pollutants (indicated by arrow 86) enter the chemostat 14 through the input port 22. Further, the nutrient mix 26 is fed to the chemostat 14 through the input port 22. In the conduit 20 of the chemostat 14, the nutrient mix 26, scrubber solution 54 and pollutants form the medium 24 for growing the algae cells 16. This medium 24 is circulated around the conduit 20 by the screw 28. Further, the conditions in the conduit 20 are maintained for maximum algal growth. For instance, in order to maintain the desired conditions, the medium 24 and the algae cells 16 are moved around the conduit 20 at a preferred fluid flow velocity of approximately fifty centimeters per second. Further, the amount of algae cells 16 in the conduit 20 is kept substantially constant. Specifically, the nutrient mix 26 and the scrubber solution 54 with pollutants 86 are continuously fed at selected rates into the conduit 20 through the input port 22, and an overflow medium 24′ containing algae cells 16 is continuously removed through the output port 30 of the conduit 20.

After entering the input port 32a of the plug flow reactor 18, the medium 24″ containing algae cells 16 moves downstream through the conduit 34 in a plug flow regime. Further, as the medium 24″ moves downstream, the modified nutrient mix 36 may be added to the conduit 34 through the input port 32b. This modified nutrient mix 36 may contain a limited amount of a selected constituent, such as nitrogen or phosphorous. The absence or small amount of the selected constituent causes the algae cells 16 to focus on energy storage rather than growth. As a result, the algae cells 16 form triglycerides.

At the end of the conduit 34, the algae separator 40 removes the algae cell concentrate 41 from the remaining effluence (indicated by arrow 88). Thereafter, the effluence 88 is discharged from the algae separator 40 through the effluence outlet 42. In order to recycle the effluence 88, it is delivered through channel 60 to the input port 50b of the scrubber 46 for reuse as the scrubber solution 54. Further, the removed algae cells (indicated by arrow 90) are delivered to the cell lysis apparatus 64. Specifically, the removed algae cells 90 pass out of the algae cell outlet 44 to the cell lysis apparatus 64. For purposes of the present invention, the cell lysis apparatus 64 lyses the removed algae cells 90 to unbind the oil therein from the remaining cell matter. After the lysing process occurs, the unbound oil and remaining cell matter, collectively identified by arrow 92, are transmitted to the oil separator 66. Thereafter, the oil separator 66 withdraws the oil from the remaining cell matter 92 as is known in the art. After this separation is performed, the oil separator 66 discharges the remaining cell matter (identified by arrow 94) out of the outlet 68 of the oil separator 66 to the input port 22 of the chemostat 14.

In the chemostat 14, the remaining cell matter 94 is utilized as a source of nutrients and energy for the growth of algae cells 16. Because small units of the remaining cell matter 94 are more easily absorbed or otherwise processed by the growing algae cells 16, the remaining cell matter 94 may first be broken down before being fed into the input port 22 of the chemostat 14. To this end, the hydrolysis apparatus 72 is interconnected between the oil separator 66 and the chemostat 14. Accordingly, the hydrolysis apparatus 72 receives the remaining cell matter 94 from the oil separator 66, hydrolyzes the received cell matter 94, and then passes hydrolyzed cell matter (identified by arrow 96) to the chemostat 14.

Referring back to the oil separator 66, it is recalled that the remaining cell matter 94 was discharged through the outlet 68. At the same time, the oil withdrawn by the oil separator 66 is discharged through the outlet 70. Specifically, the oil (identified by arrow 98) is delivered to the biofuel reactor 74. In the biofuel reactor 74, the oil 98 can be reacted with alcohol, such as methanol, to create mono-alkyl esters, i.e., biodiesel fuel. This biodiesel fuel (identified by arrow 100) is released from the exit 78 of the biofuel reactor 74 to a tank, reservoir, or pipeline (not shown) for use as fuel. Alternatively, a biofuel 100 may be synthesized in the reactor 74 and converted to jet fuel. In addition to the biofuel 100, the reaction between the oil 98 and the alcohol produces glycerin as a byproduct. For purposes of the present invention, the glycerin (identified by arrow 102) is pumped out of the exit 76 of the biofuel reactor 74 to the input port 32c of the plug flow reactor 18.

In the plug flow reactor 18, the glycerin 102 is utilized as a source of carbon by the algae cells 16. Importantly, the glycerin 102 does not provide any nutrients that may be limited to induce oil production by the algae cells 16 or to trigger flocculation. The glycerin 102 may be added to the plug flow reactor 18 at night to aid in night-time oil production. Further, because glycerin 102 would otherwise provide bacteria and/or other non-photosynthetic organisms with an energy source, limiting the addition of glycerin 102 to the plug flow reactor 18 only at night allows the algae cells 16 to utilize the glycerin 102 without facilitating the growth of foreign organisms. As shown in the FIGURE, the exit 76 of the biofuel reactor 74 may also be in fluid communication with the input port 22 of the chemostat 14 (connection shown in phantom). This arrangement allows the glycerin 102 to be provided to the chemostat 14 as a carbon source.

While the particular Transportable Algae Biodiesel System as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A portable system for producing algae cells for use in processing biofuel which comprises:
at least one enclosed chemostat formed from a plastic bladder defining a conduit for growing algae cells therein, wherein the chemostat has an input port for feeding a medium with a nutrient mix into the conduit and an output port;
a means for continuously moving the medium through the conduit at a predetermined fluid flow velocity;
a plug flow reactor formed from a plastic bladder defining a passageway having an input port for receiving algae cells from the output port of the chemostat;
a means for capturing carbon dioxide, with the capturing means delivering the carbon dioxide to each chemostat;
an algae separator in fluid communication with the passageway of the plug flow reactor for removing an algae cell concentrate from an effluence exiting the passageway; and
a means for processing biofuel from the algae cells.

2. A portable system as recited in claim 1 wherein the capturing means receives carbon dioxide created during oxidation of carbon-containing waste.

3. A portable system as recited in claim 1 wherein the capturing means receives carbon dioxide from generators.

4. A portable system as recited in claim 1 wherein the capturing means removes carbon dioxide from the atmosphere.

5. A portable system as recited in claim 1 wherein the capturing means comprises:
a scrubber having a chamber for receiving a pollutant-contaminated fluid stream; and
a scrubber solution received in the chamber for scrubbing the pollutant-contaminated fluid stream, and wherein the scrubber solution is fed to the conduit through the input port as nutrients supporting algae cell growth.

6. A portable system as recited in claim 5 further comprising a channel for recycling the effluence from the plug flow reactor to the scrubber for use as the scrubber solution.

7. A portable system as recited in claim 1 wherein the processing means comprises:
a device for lysing the algae cells removed from the conduit to unbind oil within the algae cells;
an oil separator for withdrawing the oil from remaining cell matter; and
a bioreactor for receiving the oil from the oil separator and for synthesizing biofuel from said oil.

8. A portable system as recited in claim 7 further comprising a means for recycling remaining cell matter through the input port to the conduit to support growth of algae cells with high oil content.

9. A portable system as recited in claim 7 wherein the bioreactor synthesizes glycerin from the oil, and further comprising a means for recycling the glycerin through the input port to the conduit to support growth of algae cells with high oil content.

10. A portable system as recited in claim 7 further comprising a means for adding a modified nutrient mix to the passageway in the plug flow reactor, wherein the modified nutrient mix comprises a limited amount of a selected constituent to trigger high oil production in the algae cells.

11. A portable system as recited in claim 7 wherein the lysing device uses steam to rupture the algae cells and unbind the oil therein.

12. A portable system as recited in claim 1 further comprising a means for controlling the temperature of each chemostat and the plug flow reactor.

13. A portable system for producing algae cells for use in processing biofuel which comprises:
a plastic bladder defining an endless conduit for growing algae cells therein and a passageway for receiving the algae cells from the conduit, with said bladder forming an input port for feeding a medium into the conduit and an output port for passing the algae cells out of the passageway;
a means for continuously moving the medium through the conduit at a predetermined fluid flow velocity;
a means for controlling the temperature of the medium in the bladder;
a means for capturing carbon dioxide, with the capturing means delivering the carbon dioxide to the medium;
an algae separator in fluid communication with the passageway for removing algae cell concentrate therefrom; and
a means for processing biofuel from the algae cells.

14. A portable system as recited in claim 13 wherein the processing means comprises:
a device for lysing the algae cells removed from the conduit to unbind oil within the algae cells;
an oil separator for withdrawing the oil from remaining cell matter; and
a bioreactor for receiving the oil from the oil separator and for synthesizing biofuel from said oil.

15. A portable system as recited in claim 14 further comprising a means for recycling remaining cell matter through the input port to the conduit to support growth of algae cells with high oil content.

16. A portable system as recited in claim 14 wherein the bioreactor synthesizes glycerin from the oil, and further comprising a means for recycling the glycerin through the input port to the conduit to support growth of algae cells.

17. A method for producing algae cells for use in processing biofuel which comprises the steps of:
providing a system including a chemostat formed from a plastic bladder defining a conduit for growing algae cells therein and having an input port and an output port, a plug flow reactor formed from a plastic bladder defining a passageway having an input port, and an algae separator in fluid communication with the passageway of the plug flow reactor;
transporting the system to a desired location;
feeding a medium through the input port into the conduit;
capturing carbon dioxide and delivering the carbon dioxide through the input port to the conduit;
continuously moving the medium through the conduit at a predetermined fluid flow velocity to grow algae cells in the medium;
passing algae cells from the conduit to the passageway of the plug flow reactor, with the algae cells forming an algae cell concentrate in the passageway;
controlling the temperature of each chemostat and the plug flow reactor;
removing the algae cell concentrate from the passageway of the plug flow reactor with the algae separator; and
processing biofuel from the algae cells.

18. A method as recited in claim 17 wherein the processing step comprises:
lysing the algae cells removed from the conduit to unbind oil within the algae cells;
withdrawing the oil from remaining cell matter; and
synthesizing biofuel from the oil.

19. A method as recited in claim 18 wherein the synthesizing step results in the production of glycerin, and wherein the method further comprises the step of recycling the glycerin through the input port to the conduit to support growth of algae cells.

20. A method as recited in claim 18 further comprising the step of creating the carbon dioxide by oxidizing waste.
